SEQUENCE LISTING (11 sequences)

SEQ ID NO: 1 - DNA, 2166 bp, Artificial Sequence
  Synthetic Construct
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcagtaagtatcatagatcatggagataattaaaatgataaccatctcgcaaataaataagtattttactgttttcgtaacagttttgtaataaaaaaacctataaatattccggattattcataccgtcccaccatcgggcgccttactgaatccactttgcctttctctccacaggctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggcccgtttaaacccgctgatca

SEQ ID NO: 2 - DNA, 361 bp, Artificial Sequence
  Synthetic Construct
actagaagctttattgcggtagtttatcacagttaaattgctaacgcagtcagtgcttctgacacaacagtctcgaacttaagctgcagaagttggtcgtgaggcactgggcagtaagtatcatagatcatggagataattaaaatgataaccatctcgcaaataaataagtattttactgttttcgtaacagttttgtaataaaaaaacctataaatattccggattattcataccgtcccaccatcgggcgccttactgacatccactttgcctttctctccacaggctagcatggtgagcaagggcgaggagctgttcaccggggtggtgcccatcctggtcgagctggacggcgacg

SEQ ID NO: 3 - DNA, 331 bp, Artificial Sequence
  Synthetic Construct
  misc_feature (1)..(11): n is a, c, g, or t
nnnnnnnnnnngctacgcagtcagtgcttctgacacaacagtctcgaacttaagctgcagaagttggtcgtgaggcactgggcagtaagtatcatagatcatggagataattaaaatgataaccatctcgcaaataaataagtattttactgttttcgtaacagttttgtaataaaaaaacctataaatattccggattattcataccgtcccaccatcgggcgccttactgacatccactttgcctttctctccacaggctagcatggtgagcaagggcgaggagctgttcaccggggtggtgcccatcctggtcgagctggacggcgaa

SEQ ID NO: 4 - DNA, 327 bp, Artificial Sequence
  Synthetic Construct
  misc_feature (11)..(11): n is a, c, g, or t
  misc_feature (305)..(305): n is a, c, g, or t
  misc_feature (316)..(319): n is a, c, g, or t
  misc_feature (321)..(327): n is a, c, g, or t
gttagaagctntatgcggtagtttatcacagttaaattgctaacgcagtcagtgcttctgacacaacagtctcgaacttaagctgcagaagttggtcgtgaggcactgggcagtaagtatcatagatcatggagataattaaaatgataaccatctcgcaaataaataagtattttactgttttcgtaacagttttgtaataaaaaaacctataaatattccggattattcataccgtcccaccatcgggcgccttactgacatccactttgcctttctctccacaggctagcatggtgagcaanggcgaggagcnnnncnnnnnnn

SEQ ID NO: 5 - DNA, 355 bp, Artificial Sequence
  Synthetic Construct
tagaagcttatgcggtagtttatcacagttaaattgctaacgcagtcagtgcttctgacacaacagtctcgaacttaagctgcagaagttggtcgtgaggcactgggcagtaagtatcatagatcatggagataattaaaatgataaccatctcgcaaataaataagtattttactgttttcgtaacagttttgtaataaaaaaacctataaatattccggattattcataccgtcccaccatcgggcgccttactgacatccactttgcctttctctccacaggctagcatggtgagcaagggcgaggagctgttcaccggggtggtgcccatcctggtcgagctggacggcga

SEQ ID NO: 6 - DNA, 184 bp, Artificial Sequence
  Synthetic Construct
tagaagctttattgcggtagtttatcacagttaaattgctaacgcagtcagtgcttctgacacaacagtctcgaacttaagctgcagaagttggtcgtgaggcactgggcagctagcatggtgagcaagggcgaggagctgttcaccggggtggtgcccatcctggtcgagctggacggcgacg

SEQ ID NO: 7 - DNA, 155 bp, Artificial Sequence
  Synthetic Construct
  misc_feature (1)..(14): n is a, c, g, or t
nnnnnnnnnnnnnncgcagtcagtgcttctgacacaacagtctcgaacttaagctgcagaagttggtcgtgaggcactgggcagctagcatggtgagcaagggcgaggagctgttcaccggggtggtgcccatcctggtcgagctggacggcgaa

SEQ ID NO: 8 - DNA, 153 bp, Artificial Sequence
  Synthetic Construct
  misc_feature (1)..(8): n is a, c, g, or t
nnnnnnnngctacgcagtcagtgcttctgacacaacagtctcgaacttaagctgcagaagttggtcgtgaggcactgggcagctagcatggtgagcaagggcgaggagctgttcaccggggtggtgcccatcctggtcgagctggacggcgaa

SEQ ID NO: 9 - DNA, 152 bp, Artificial Sequence
  Synthetic Construct
  misc_feature (130)..(131): n is a, c, g, or t
  misc_feature (136)..(137): n is a, c, g, or t
  misc_feature (141)..(142): n is a, c, g, or t
  misc_feature (144)..(152): n is a, c, g, or t
ttagaagctttattgcggtagtttatcacagttaaattgctaacgcagtcagtgcttctgacacaacagtctcgaacttaagctgcagaagttggtcgtgaggcactgggcagctagcatggtgagcaanngcgannagcnncnnnnnnnnn

SEQ ID NO: 10 - DNA, 151 bp, Artificial Sequence
  Synthetic Construct
  misc_feature (140)..(151): n is a, c, g, or t
ttagaagctttattgcggtagtttatcacagttaaattgctaacgcagtcagtgcttctgacacaacagtctcgaacttaagctgcagaagttggtcgtgaggcactgggcagctagcatggtgagcaagggcgagagcnnnnnnnnnnnn

SEQ ID NO: 11 - DNA, 182 bp, Artificial Sequence
  Synthetic Construct
tagaagctttattgcggtagtttatcacagttaaattgctaacgcagtcagtgcttctgacacaacagtctcgaacttaagctgcagaagttggtcgtgaggcactgggcagctagcatggtgagcaagggcgaggagctgttcaccggggtggtgcccatcctggtcgagctggacggcga

(19) United States
(12) Patent Application Publication
(10) Pub. No.: US 2022/0251600 A1
(43) Pub. Date: Aug. 11, 2022

(54) RECOMBINANT TRANSFER VECTORS FOR PROTEIN EXPRESSION IN INSECT AND MAMMALIAN CELLS

(71) Applicant: X-Chem, Inc., Waltham, MA (US)

(72) Inventors: Alexander LITOVCHICK, Sudbury, MA (US); Ragunath CHANDRAN, Waltham, MA (US); Moritz VON RECHENBERG, Waltham, MA (US); John CUOZZO, Natick, MA (US)

(21) Appl. No.: 17/622,335

(22) PCT Filed: Jun. 25, 2020
(86) PCT No.: PCT/US20/39584

§ 371 Date: Dec. 23, 2021

Related U.S. Application Data
(60) Provisional application No. 62/867,468, filed on Jun. 27, 2019.

(51) Int. Cl. C12N 15/85

(52) U.S. Cl. CPC C12N 15/85; C12N 2710/14122; C12N 2800/50; C12N 2830/50; C12N 2830/42; C12N 2830/48; C12N 2800/90

(57) ABSTRACT

Described herein are recombinant vectors and methods for their use in expressing recombinant proteins in both insect and mammalian cells. The invention is based on recombinant transfer vectors that enable expression of one or more transgenes to be directed by an insect cell-competent promoter and a mammalian cell-competent promoter, both present within a single expression cassette in the vector, and active conditional on the host cell.

### SEQUENCE LISTING

The instant application contains a Sequence Listing which has been submitted electronically in ASCII format and is hereby incorporated by reference in its entirety. Said ASCII copy, created on Jun. 24, 2020, is named 50719-059WO2_Sequence_Listing_6_24_20_ST25 and is 8,864 bytes in size.

### FIELD OF THE INVENTION

The invention relates to methods and compositions for recombinant vectors for use in expressing proteins in insect and mammalian cells.

### BACKGROUND

Gene transfer vectors have been employed as a powerful tool for transgene delivery and expression in research, biotechnology, and clinical applications. Such vectors facilitate the insertion of single or multiple genes into expression cassettes for heterologous production of proteins in target cells. Given the importance of recombinant expression systems, there exists a need for improved transfer vectors that enable transgene expression in multiple host organisms.

### SUMMARY OF THE INVENTION

The present disclosure provides methods and compositions for expressing recombinant proteins in insect and mammalian cells. The invention is based on recombinant transfer vectors that enable expression of one or more (e.g., 1, 2, 3, 4, or more) transgenes to be directed by an insect cell-competent promoter and a mammalian cell-competent promoter, both present within a single expression cassette in the vector, and active conditional on the host cell (e.g., an insect cell or a mammalian cell). Also described herein are methods for expressing recombinant proteins using the vectors described herein or recombinant viruses produced from said vectors.

In a first aspect, the invention provides a recombinant DNA vector including in a 5′ to 3′ direction: (a) a mammalian cell-competent promoter, (b) a non-coding exon operably linked to an artificial intron, the artificial intron comprising a splice donor sequence, an insect cell-competent promoter, a splice branch point, a polypyrimidine tract, and a splice acceptor sequence, and (c) one or more (e.g., 1, 2, 3, 4, or more) transgenes operably linked to the mammalian cell-competent promoter and to the insect cell-competent promoter.

In some embodiments, the mammalian cell-competent promoter is selected from the group including a cytomegalovirus (CMV) enhancer/promoter, simian virus 40 (SV40) promoter, CAG promoter, elongation factor 1 (EF1-α) promoter, phosphoglycerate kinase 1 (PGK1) promoter, β-actin promoter, early growth response 1 (EGR1) promoter, eukaryotic translation initiation factor 4A1 (eIF4A1) promoter, glyceraldehyde 3-phosphate dehydrogenase (GAPDH) promoter, human immunodeficiency virus long terminal repeat (HIV LTR) promoter, Adenoviral promoter, and Rous Sarcoma Virus (RSV) promoter. In some embodiments, the mammalian cell-competent promoter is a CMV enhancer/promoter.

In some embodiments, the insect cell-competent promoter is selected from a group including a polyhedrin (PH) promoter, heat shock protein (HSP) promoter, p6.9 promoter, p9 promoter, p10 promoter, actin 5c (Ac5) promoter, Orgyia pseudotsugata multicapsid nuclear polyhedrosis virus immediate early-1 (OpIE1) promoter, Orgyia pseudotsugata multicapsid nuclear polyhedrosis virus immediate early-2 (OpIE2) promoter, and an immediate early-0 (IE0) promoter. In some embodiments, the insect-cell competent promoter is a PH promoter.

In some embodiments the vector further includes a 5′ untranslated region (5′ UTR) with a Kozak sequence.

In some embodiments, the vector further includes a 3′ untranslated region (3′ UTR). In some embodiments, the 3′ UTR includes an enhancer sequence. In some embodiments, the enhancer sequence is a Woodchuck Hepatitis Virus Posttranscriptional Regulatory Element (WPRE). In some embodiments, the 3′ UTR further includes one or more terminator sequences. In some embodiments, the one or more terminator sequences is selected from a group including a bovine growth hormone (bGH) terminator sequence and a SV40 terminator sequence.

In some embodiments, the vector further includes one or more nucleic acid sequences encoding one or more selectable marker genes. In some embodiments, the one or more selectable marker genes are selected from the group including an ampicillin resistance gene, gentamycin resistance gene, carbenicillin resistance gene, chloramphenicol resistance gene, kanamycin resistance gene, nourseothricin resistance gene, tetracycline resistance gene, zeocin resistance gene, streptomycin resistance gene, and spectinomycin resistance gene.

In some embodiments, the vector further includes two translocation elements. In some embodiments, the two translocation elements are bacterial transposon Tn7R and Tn7L translocation elements.

In some embodiments, the one or more (e.g., 1, 2, 3, 4, or more) transgenes are mammalian genes. In some embodiments, the one or more (e.g., 1, 2, 3, 4, or more) transgenes are insect genes.

In another aspect, the invention provides a method of expressing a recombinant protein in a host cell, the method including contacting the host cell with the vector of any one of the foregoing aspects and embodiments; and expressing the recombinant protein in the host cell. In some embodiments, the host cell is a mammalian cell.

In yet another aspect, the invention provides a method of expressing a recombinant protein in a host cell, the method including contacting the host cell with a recombinant virus produced using the vector of any one of the foregoing aspects and embodiments; and expressing the recombinant protein in the host cell. In some embodiments, the host cell is an insect cell or a mammalian cell.

### BRIEF DESCRIPTION OF THE DRAWINGS

FIGS. 1A-1C show a series of schematic diagrams demonstrating a transfer vector for use in the production of recombinant proteins in insect and mammalian cells. FIG. 1A shows a schematic of an exemplary transfer vector, outlining the individual elements of this exemplary gene expression cassette, including a 5′ untranslated region (5′ UTR) with a Kozak sequence, initiation codon ATG, gene-coding sequence, which is instantiated as an emerald green fluorescent protein (GFP) model protein, followed by the stop codon TAA and then by a 3′ UTR expression enhancer Woodchuck Hepatitis Virus Posttrranscriptional Regulatory Element (WPRE) and bovine growth hormone (bGH) and simian virus 40 (SV40) polyadenylation signals. Upstream of the 5′ UTR is an artificial intron including the PH promoter, upstream of which lies a non-coding mini-exon sequence, and further upstream, the CMV enhancer/promoter. Other elements of this vector include translocation sites Tn7L and Tn7R, gentamycin and ampicillin resistance genes, and the E. coli origin of replication. FIG. 1B shows a schematic of mRNA produced from the transfer vector of FIG. 1A in insect cells. FIG. 1C shows a schematic of mRNA produced from the same vector in mammalian cells.

FIGS. 2A-2B show a series of fluorescence images demonstrating dose-dependent expression of a GFP transgene in insect and mammalian cells infected with a recombinant vector of the invention. Cultures of insect SF9 cells (FIG. 2A) and mammalian HEK293F cells (FIG. 2B) were infected with viral particles harboring a genome represented by the vector in FIG. 1A at varying doses. Arabic numerals correspond to the viral dosing regimen (e.g., 1=no virus control; 2=200 uL virus; 3=400 uL virus). Fluorescence images were obtained 16 hours post-infection and demonstrate robust and dose-dependent GFP expression in both insect and mammalian cells infected by the same recombinant virus.

FIG. 3 shows an image of a 4% agarose gel, stained with Ethidium Bromide, demonstrating a splicing event in the transcript produced by the vector presented in FIG. 1A in mammalian cells. Cultured HEK293 cells were infected with a recombinant viral vector containing a GFP transgene. Total RNA was extracted, and reverse transcription was performed followed by PCR amplification. As a control, PCR amplification was also performed from the plasmid only. Expected length of the spliced product was 186 bp, whereas un-spliced precursor (as in the plasmid) was 357 bp long. Both RT-PCR reactions with gene-specific (lane 2) and oligo-dT/random hexamers (lane 3) were spliced, and their lengths were about 180-190 bp on the gel, as expected if the intron was removed. Amplification of the plasmid produced a 350 bp product, as expected if the intron was present (lane 4).

FIGS. 4A-4B show a series of images demonstrating sequencing alignment of the recombinant vector and the mRNA transcript produced from the vector. Consistent with the results shown in FIG. 3, Sanger sequencing (Genewiz) of PCR products of the vector alone (FIG. 4A) or the mRNA product produced by the vector in HEK293 cells (FIG. 4B) confirmed the predicted lengths of the full vector and the spliced mRNA transcript.

### DEFINITIONS

As used herein, the term “artificial” means non-naturally occurring. For example, an intron sequence may be considered artificial when it is modified (e.g., substituted, inserted, concatenated, or flanked) with recombinant nucleotide sequences, such as a nucleotide sequence including a polyhedrin (PH) promoter, in such a way that the modified sequence is not found occurring in nature. A non-limiting example of an artificial intron sequence includes an intron having, in a 5′ to 3′ direction, a splice donor sequence, a heterologous promoter (e.g., an insect cell-competent promoter or a strong promoter, such as a PH promoter), a splice branch point, a polypyrimidine tract, and a splice acceptor sequence.

As used herein, the terms “3′ untranslated region” and “3′ UTR” refer to the region 3′ with respect to the stop codon of an mRNA molecule. The 3′ UTR is not translated into protein, but includes regulatory sequences important for polyadenylation, localization, stabilization, and/or translation efficiency of an mRNA. Regulatory sequences in the 3′ UTR may include enhancers, silencers, AU-rich elements, poly-A tails, terminators, and microRNA recognition sequences. The terms “3′ untranslated region” and “3′ UTR” may also refer to the corresponding regions of the gene encoding the mRNA molecule.

As used herein, the term “5′ untranslated region” and “5′ UTR” refer to a region of an mRNA molecule that is 5′ with respect to the start codon. This region is essential for the regulation of translation initiation. The 5′ UTR can be entirely untranslated or may have some of its regions translated in some organisms. The transcription start site marks the start of the 5′ UTR and ends one nucleotide before the start codon. In eukaryotes, the 5′ UTR includes a Kozak consensus sequence harboring the AUG start codon. The 5′ UTR may include cis-acting regulatory elements also known as upstream open reading frames that are important for the regulation of translation. This region may also harbor upstream AUG codons and termination codons. Given its high GC content, the 5′ UTR may form secondary structures, such as hairpin loops that play a role in the regulation of translation.

As used herein, the terms “baculovirus” and “baculoviral” refer to double-stranded DNA viruses from the baculoviridae family of viruses known to infect arthropods, lepidoptera, hymenoptera, diptera, and decapoda. These terms may refer to the wild-type or recombinant baculoviral genome, viral particles (e.g., virions), and/or baculoviral-derived DNA or protein. Naturally-occurring baculoviruses are known to largely target invertebrates (e.g., insects) and despite having the capacity to enter mammalian cells in cell culture, cannot naturally replicate therein.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common organism (e.g., insect cells or mammalian cells), a common tissue (e.g., epithelial tissue, neural tissue, connective tissue, or muscle tissue), and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume.

As used herein, the term “express” refers to one or more of the following events: (1) production of an RNA primary transcript from a DNA sequence by transcription; (2) processing of an RNA transcript into mature mRNA (e.g., by splicing, editing, 5′ cap formation, and/or 3′ end processing); (3) translation of an mRNA into a polypeptide or protein; and (4) post-translational modification of a polypeptide or protein.

As used herein, the term “exon” refers to a region of a gene which is preserved in the mature mRNA after splicing (e.g., in the 5′ UTR). Primary RNA transcripts contain both exons and introns. Introns are further spliced out and only exons are included in the mature mRNA following processing of the primary transcript. Sequences of some exons are translated into protein, wherein the sequence of the exon determines the amino acid composition of the protein. Some exons that are included in the mature mRNA may be non-coding (e.g., in the 5′ and/or 3′ UTR).

As used herein, the term “intron” refers to a region of a gene, the nucleotide sequence of which is excised out, or spliced during mRNA maturation. The term intron also refers to the corresponding region of the RNA transcribed from a gene. Introns together with exons are transcribed into a primary RNA transcript, but are further removed by splicing, and are not included in the mature mRNA. Two types of splicing mechanisms are known: 1) a spliceosomal process assisted by small nuclear ribonucleoproteins; and 2.) self-splicing. An intron subjected to spliceosomal splicing typically includes a 5′ splice donor site, and a splice acceptor site at the 3′ end of the intron along with other regulatory sequences such as a branch point, and a polypyrimidine tract. As used herein, the term “intron” may also refer to an artificial intron (e.g., non-naturally occurring) which is constructed by inserting regulatory sequences such as splice donor sequences, acceptor sequences, a branch point, and a polypyrimidine tract targeted for recognition by spliceosomes into a DNA construct to be expressed in a host cell. A non-limiting example of an artificial intron includes a nucleotide sequence having, in a 5′ to 3′ direction, a 5′ splice donor site, a sequence targeted for splicing (e.g., a heterologous promoter sequence, such as, for example, a polyhedrin promoter sequence), a branch point, a polypyrimidine tract, and a 3′ splice acceptor site.

As used herein, the term “heterologous” refers to a nucleic acid sequence that is not normally contained within a specific DNA or RNA molecule, not normally expressed in a cell (e.g., a mammalian cell or an insect cell), and/or is not normally found occurring in nature. As used herein, a heterologous nucleic acid may, for example, be a promoter sequence, an artificial intron, a non-coding exon, a transgene, or any associated regulatory sequences individually or in combination. Furthermore, the term “heterologous” may also refer to an amino acid sequence of a protein that is not normally expressed in a cell (e.g., a mammalian cell or an insect cell), and/or is not normally found occurring in nature.

As used herein, the terms “host” and “host cell” refer to any prokaryotic or eukaryotic organism (e.g., mammalian, invertebrate, bacterial, and avian, among others) capable of infection by the vectors described herein. These terms may refer to wild-type hosts or hosts infected with a recombinant vector of the instant invention.

As used herein, the terms “infect” and “infection” refer to the process by which viral particles (e.g., virions) invade and enter host cells (e.g., insect cells, mammalian cells). Generally, this process can be divided into several stages including cell attachment, penetration, uncoating, replication, assembly, and release. During the attachment phase, a viral particle binds to host's cell surface receptors via viral capsid proteins. Receptor attachment results in the penetration phase during which the viral particle is internalized by endocytosis, micropinocytosis, or fusion with the cell membrane of the host. Once inside the cell, the viral particles shed their capsid proteins during the process of uncoating, thereby releasing their genome inside of the host cell. If the virus is competent to replicate within the cellular context of the host cell, the replication phase may occur. During this phase, the viral genome replicates its RNA-based or DNA-based genome, a process that may require the synthesis and assembly of viral proteins. In the subsequent assembly phase, the newly synthesized viral proteins assemble into new viral particles (e.g., virions) and may undergo posttranslational modification. In the final release phase, the viral particles acquire their viral envelope by adopting and modifying parts of the host cell membrane. During this final stage, the viral particles escape the host cell by cell lysis.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present. As a non-limiting example, an exon and an intron in a primary RNA transcript or in a DNA sequence encoding said transcript may be operably linked to one another if the exon facilitates splicing out of the intron.

As used herein, the term “monocistronic” refers to an RNA or DNA construct that includes the coding sequence for a single protein or polypeptide product.

As used herein, the term “plasmid” refers to a to an extrachromosomal circular double stranded DNA molecule into which additional DNA segments may be inserted (e.g., ligated). A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal plasmids). Other plasmids (e.g., non-episomal vectors) can be integrated into the genome of a host cell upon introduction into the host cell, and thereby are replicated along with the host genome. Certain plasmids are capable of directing the expression of genes to which they are operably linked.

As used herein, the term “polycistronic” refers to an RNA or DNA construct that includes the coding sequence for at least two protein or polypeptide products.

As used herein, the term “polypyrimidine tract” refers to a region of an intron that is about 5-40 nucleotides upstream (e.g. 5′) to the splice acceptor site and typically contains 15-20 pyrimidine nucleotides (e.g. C and T/U). The polypyrimidine tract functions during splicing by facilitating the organization of the splicesome.

As used herein, the term “promoter” refers to a recognition site on DNA that is bound by an RNA polymerase. The polymerase drives transcription of the transgene. The promoter may be a “mammalian cell-competent promoter,” meaning that the promoter is capable of driving gene expression in a mammalian cell. A mammalian-cell competent promoter may be competent in mammalian cells only or may be competent in mammalian cells and other cell types. The promoter may also be an “insect cell-competent promoter,” meaning that the promoter is capable of driving gene expression in an insect cell. An insect cell-competent promoter may be competent in insect cells only or may be competent in insect cells and other cell types. The promoter may be a strong promoter or a weak promoter, depending on its affinity for RNA polymerase and/or sigma factor, its rate of transcription initiation, and its levels of transcription. The strength of a promoter is related to the similarity of the promoter nucleotide sequence to the ideal consensus sequence of the RNA polymerase. A strong promoter exhibits frequent and strong binding of RNA polymerase, high levels of transcription and, consequently, high levels of the transcript under its control. Promoter strength may be determined by comparing levels of RNA expression under its control with respect to a reference promoter (e.g., an adenoviral promoter, simian virus 40 (SV40) promoter, or a human immunodeficiency virus long terminal repeat (HIV LTR) promoter, among others) in a particular host cell type having a specified level of RNA expression. A promoter that drives expression of a transgene equal to or higher than the expression level driven by a reference promoter within a particular cell-type may be considered a strong promoter. Non-limiting examples of strong promoters include the CMV enhancer/promoter, EF1-α promoter, and CAG promoter, PH promoter, and the Ac5 promoter. A weak promoter exhibits infrequent and/or weak binding of RNA polymerase, low levels of transcription, and consequently, low levels of the transcript under its control. Non-limiting examples of weak promoters include the ubiquitin C promoter and phosphoglycerate kinase 1 promoter. Additionally, the term “promoter” may refer to a synthetic promoter, which is a regulatory DNA sequence that does not occur naturally in a biological system. Synthetic promoters include parts of naturally occurring promoters combined with polynucleotide sequences that do not occur in nature and can often be optimized to express recombinant DNA using a variety of transgenes, vectors, and target cell types. One of skill in the art will appreciate that promoter strength may depend on the particular cell type, tissue, and organism in which the promoter is active.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “regulatory sequence” includes promoters, enhancers, terminators, and other expression control elements (e.g., polyadenylation signals) that control the transcription or translation of a gene. Such regulatory sequences are described, for example, in Goeddel, Gene Expression Technology: Methods in Enzymology 185 (Academic Press, San Diego, Calif., 1990); incorporated herein by reference.

As used herein, the term “selectable marker” and “selectable marker gene” refer to a gene that is introduced into a cell in order to facilitate the selection of cells. For example, one or more selectable marker may be introduced into a recombinant vector described herein to allow for selection of cells containing the vector. Selectable markers may be antibiotic resistance genes, such as, for example, an ampicillin resistance gene, a gentamycin resistance gene, a carbenicillin resistance gene, a chloramphenicol resistance gene, a kanamycin resistance gene, or nourseothricin resistance gene.

As used herein, the terms “splice acceptor sequence” or “splice acceptor site” refer to a DNA or RNA sequence at the 3′ end of an intron that is necessary for splicing out introns from a primary transcript. The splice acceptor sequence typically ends with an invariant AG sequence.

As used herein, the term “splice branch point” refers to a region of the intron that includes an adenine nucleotide necessary for splicing out introns from a primary transcript. The splice branch point is critical for lariat formation that occurs within the intron during splicing. The splice branch point is typically positioned within 20-50 nucleotides upstream of (e.g. 5′ to) the splice acceptor sequence.

As used herein, the terms “splice donor sequence” or “splice donor site” refer to a DNA or RNA nucleotide sequence at the 5′ end of an intron that is necessary for splicing out introns from a primary transcript. The splice donor sequence typically is an invariant GU sequence at the 5′ end of the intron.

As used herein, the terms “terminator” and “terminator sequence” refer to a DNA or RNA nucleotide sequence that marks the end of a transcriptional unit (e.g. a gene or a transgene) and initiates the release of newly synthesized RNA from the ensemble of transcriptional proteins. Terminators are found downstream of (e.g. 3′ to) the gene of interest and downstream of 3′ regulatory elements. Terminator sequences contribute to the half-life of the RNA molecule, and consequently to levels of gene expression.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the terms “transduction” and “transduce” refer to a method of introducing a vector construct or a part thereof into a cell. Wherein the vector construct is included in a viral vector, such as for example an AAV vector, transduction refers to viral infection of the cell and subsequent transfer and/or integration of the vector construct or part thereof into the cell genome.

As used herein, the term “transgene” refers to a recombinant nucleic acid (e.g., DNA or cDNA) encoding a gene product (e.g., a recombinant protein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the term “vector” includes a biological vehicle for the transfer of nucleic acids, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Expression vectors described herein may include a polynucleotide sequence as well as, e.g., additional sequence elements used for the expression of proteins and/or the integration of these polynucleotide sequences into the genome of a cell. Certain vectors that can be used for the expression of a transgene as described herein include vectors that include regulatory sequences, such as promoter and enhancer regions, which direct gene transcription. Other useful vectors for recombinant gene expression include polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors described herein may also include polynucleotides encoding one or more markers for selection of cells that include such a vector. Non-limiting examples of suitable markers include genes that encode resistance to antibiotics, such as ampicillin, gentamicin, chloramphenicol, kanamycin, nourseothricin, carbenicillin, tetracycline, zeocin, streptomycin, or spectinomycin. The term “vector” may also refer to a shuttle vector or a transfer vector. A shuttle vector is a type of vector, such as a plasmid, constructed in a way that enables it to propagate in two different host species, thereby facilitating manipulation in two or more different cell types. Shuttle vectors may be used for amplification of a heterologous gene in a first host cell type (e.g., E. coli cells) for expression in a second host cell type (e.g., insect or mammalian cells). A transfer vector is a vector, such as a plasmid, that incorporates heterologous nucleic acid sequences for delivery to target cells.

As used herein, the term “wild-type” refers to a genotype with the highest frequency for a particular gene in a given organism.

### DETAILED DESCRIPTION

Described herein are compositions and methods that allow for expression of recombinant proteins in insect and mammalian cells. The present invention is based on recombinant transfer vectors (e.g. plasmids) that accommodate insertion of single or multiple genes for protein expression in multiple host cell types (e.g., mammalian cells and insect cells). The vectors facilitate preparation of recombinant viral particles capable of driving protein expression in both mammalian and insect cells. Such viral particles may be used according to the methods of the present invention to infect host cells under conditions that allow for infection of the cells with virus and the production of recombinant proteins. Additionally, the vectors of the present invention can be used to transiently drive protein expression in host cells by contacting the cells with the vector under conditions that allow vector entry and subsequent expression of recombinant proteins.

The present invention facilitates expression of recombinant proteins in both insect and mammalian cells by providing a transfer vector containing an expression cassette in which the transgene of interest is inserted downstream (e.g. 3′ to) an insect cell-competent promoter and a mammalian cell-competent promoter, both positioned upstream of (e.g. 5′ to) the transgene of interest and oriented in the same direction within the cassette. The insect cell-competent promoter drives transgene expression in insect cells, but not mammalian cells, whereas the mammalian cell-competent promoter drives transgene expression in mammalian cells, but not insect cells. Such a vector allows for gene expression to be differentially controlled by two different promoters conditional on the host cell.

Furthermore, the promoter configuration utilized in the vectors of the invention is unique, and facilitates efficient gene expression in both host cell types. Specifically, the vector design features the placement of the insect cell-competent promoter into an artificial intron immediately downstream (e.g., 3′) from a non-coding exon (e.g., a non-coding mini-exon), which is in turn placed immediately downstream from the mammalian cell-competent promoter. This configuration enables transgene expression in insect cells to be regulated directly by the insect cell-competent promoter without interference from the mammalian cell-competent promoter. Transcripts produced in mammalian cells from the mammalian cell-competent promoter include an insect-cell competent promoter that is removed during RNA splicing as a result of its insertion into the artificial intron. This vector design ensures that the insect-cell competent promoter does not interfere with translation in mammalian cells.

In one particular vector design, the artificial intron containing the insect cell-competent promoter is created by flanking the insect cell-competent promoter with a splice donor sequence on its 5′ end, and, in a 5′ to 3′ direction, a splice branch point, polypyrimidine tract, and splice acceptor sequences on its 3′ end. The transgene selected for expression in mammalian and insect cells is positioned downstream of the insect cell-competent promoter, the transgene being flanked on its 5′ end by the 5′ untranslated region (5′ UTR) having a Kozak sequence and the start codon (e.g., ATG), and on its 3′ end, in a 5′ to 3′ direction, by a stop codon (e.g., TAG, TAA, or TGA), a 3′ untranslated region (3′ UTR) and optional regulatory sequences, including but not limited to enhancer sequences, terminator sequences, poly-A tail, among others. The vectors of the present invention may also include nucleic acid sequences encoding one or more selectable markers, such as antibiotic resistance genes, as well as translocation elements, and an origin of replication sequence.

### Intron Sequence Elements

The vectors of the invention allow for the expression of single or multiple transgenes from a single expression cassette using two promoters oriented in the same direction within the cassette. The first promoter may, for example, be active only in mammalian cells (e.g., a mammalian cell-competent promoter), while the second promoter may be, for example, active only in insect cells (e.g., an insect cell-competent promoter). When introduced into mammalian cells, the primary transcript produced from this vector is driven by the first promoter and includes the second promoter within the transcript. To avoid translational interference from the potential presence of unproductive start codons and/or premature stop codons within the second promoter, the present invention provides artificial intron sequence elements within the vector to remove the second promoter from the primary transcript by a splicing event. Specifically, the recombinant vectors described herein incorporate the second promoter into an artificial intron that can be spliced out once the vector is transcribed within a cell. The artificial intron includes the second promoter flanked on its 5′ end by a splice donor sequence and on its 3′ end by, in a 5′ to 3′ direction, a splice branch point, polypyrimidine tract, and splice acceptor sequence. Positioned immediately upstream of the artificial intron and immediately downstream of the first promoter is a non-coding exon (e.g. a non-coding mini-exon) that facilitates splicing out of the artificial intron. The non-coding exon may include any nucleic acid sequence that does not contain regulatory elements or an AUG start codon. Sequences that may be contained within a non-coding exon include, for example, a Kozak sequence. The non-coding exon is not translated into protein and has little or no effect on protein translation of the transgene in the expression cassette of the vector described herein. Within the context of the vector of the invention, the non-coding exon is positioned upstream of the artificial intron in order to facilitate removal of the intron by RNA splicing.

### Promoters

The vectors of the present inventions include insect cell-competent and mammalian cell-competent promoter sequences operably linked to a nucleic acid sequence encoding single or multiple transgenes of interest within a single expression cassette. Mammalian cell-competent promoters are capable of binding mammalian RNA polymerase proteins and driving gene transcription only in mammalian cells. Conversely, insect cell-competent promoters are capable of controlling gene expression only in insect cells.

Exemplary mammalian cell-competent promoters include, but are not limited to a cytomegalovirus (CMV) enhancer/promoter, simian virus 40 (SV40) promoter, CAG promoter, elongation factor 1 (EF1-α) promoter, phosphoglycerate kinase 1 (PGK1) promoter, β-actin promoter, early growth response 1 (EGR1) promoter, eukaryotic translation initiation factor 4A1 (eIF4A1) promoter, glyceraldehyde 3-phosphate dehydrogenase (GAPDH) promoter, human immunodeficiency virus long terminal repeat (HIV LTR) promoter, Adenoviral promoter, or a Rous Sarcoma Virus (RSV) promoter, among others.

Non-limiting examples of insect cell-competent promoters include a polyhedrin (PH) promoter, heat shock protein (HSP) promoter, p6.9 promoter, p9 promoter, p10 promoter, actin 5c (Ac5) promoter, Orgyia pseudotsugata multicapsid nuclear polyhedrosis virus immediate early-1 (OpIE1) promoter, Orgyia pseudotsugata multicapsid nuclear polyhedrosis virus immediate early-2 (OpIE2) promoter, immediate early-0 (IE0) promoter among others. Exemplary insect-cell competent promoters are described in Lin et al. J. Biotechnol. 165(1): 11-17 (2013), the disclosure of which is herein incorporated by reference in its entirety. One of skill in the art would recognize that other mammalian cell-competent and insect cell-competent promoters may also be suitable for use with the invention.

Promoters suitable for use in conjunction with the invention may be strong promoters. Promoter strength is classified on the basis of its affinity for RNA polymerase, rate of transcription initiation, and level of expression of the primary transcript. Non-limiting examples of strong promoters include the CMV promoter, EF1-α promoter, and CAG promoter, PH promoter, Ac5 promoters, Adenoviral promoter, SV40 promoter, and HIV LTR promoter. Alternatively, the invention may employ weak promoters which are established and well-known known in the art.

### Transgene Expression

The vectors described herein may be used to deliver and express one or more (e.g., 1, 2, 3, 4, or more) transgenes of interests into a host cell (e.g., an insect cell and/or a mammalian cell). In some embodiments, the vector of the present invention includes a monocistronic expression cassette for expression of a single transgene. Accordingly, the vectors described herein may include a polynucleotide encoding a transgene of interest flanked on the 5′ by the start codon and the 5′ UTR and on the 3′ end by a stop codon and the 3′ UTR. In applications directed to the expression of two or more (e.g., 2, 3, 4, or more) transgenes in a polycistronic expression cassette from a single vector of the invention, the two or more transgenes may be separated from one another by one or more (e.g., 1, 2, 3, or more) nucleic acid sequences encoding 2A self-cleaving peptides (e.g., T2A, P2A, E2A, or F2A self-cleaving peptides). Exemplary methods of use of nucleic acid sequences encoding 2A self-cleaving peptides for use in polycistronic expression cassettes are provided in Liu et al, Sci. Rep. 7(1): 2193 (2017), the disclosure of which is incorporated by reference in its entirety. The incorporation of 2A self-cleaving peptide-encoding sequences into the vectors of the invention may be performed according to methods well-known to one of skill in the art.

The transgene of interest may encode a protein suitable for expression in insect and mammalian cells. In some embodiments, the transgene is heterologous with respect to the vector described herein. In some embodiments, the transgene is heterologous with respect to the host cell. Generally and without limitation, the transgenes may encode proteins belonging to a protein class that includes kinases, phosphatases, proteases, lipases, ligases, transferases, glycosylases, nucleases, polymerases, hydrolases, isomerases, synthases, GTPases, ATPases, deaminases, cytokines, ubiquitinases, deubiquitinases, transmembrane receptors, transcription factors, RNA binding proteins, DNA binding proteins, E3-ligases, secreted proteins, cytoskeletal proteins, oxidases, reductases, and protein-protein interaction targets, among others. In some embodiments, the transgenes encode membrane proteins. In some embodiments, the membrane proteins are membrane receptors, transport proteins, membrane enzymes, and/or cell adhesion proteins. In some embodiments, the membrane proteins are glycoproteins, G-protein coupled receptors, nuclear receptors, ion channels, and/or ATP-binding cassette drug transporters, among others. The transgenes suitable for use with the vectors of the invention may also encode chromatin remodeling proteins, antibacterial proteins, and/or ubiquitin ligase proteins. Transgenes suitable for use with the invention may also include protein tags such as, for example, maltose-binding protein tag, SNAP tag, FLAG tag, 6×His-tag, HaloTag, and fluorescent protein tags, among others. Other examples of transgenes for use with the vectors of the invention include chimeric proteins, such as, for example glutathione S-transferase fusion proteins, chimeric antibodies, among others.

The transgenes suitable for use with the vectors described herein may also be reporter genes useful for determining the efficacy of the vector to drive protein expression. In some embodiments the reporter genes are green fluorescent protein (GFP), yellow fluorescent protein (YFP), blue fluorescent protein (BFP), cyan fluorescent protein (CFP), red fluorescent protein (RFP), mCherry, dsRed, luciferase (Luc) and δ-galactosidase (lacZ), chloramphenicol acetyltransferase (CAT), among others. One of skill in the art would appreciate that other reporter genes may be suitable for use in conjunction with the present invention.

The transgenes suitable for expression via the vectors described herein may encode protein domains that can function independently of the rest of the protein chain. Such protein domains may organize into a stable three-dimensional structure with or without the help of molecular chaperones. Protein domains may have varying lengths including, but not limited to ranges between 50 to 250 amino acids. For a detailed description of chain lengths in protein domains, see, for example Xu et al. Folding and Design 3(1):11-7 (1998), the disclosure of which is herein incorporated by reference. Non-limiting examples of protein domains include ligand-binding domains, DNA-binding domains, RNA-binding domains, binding partner-binding domains, deaminase domains, ion-binding domains (e.g., Ca2+-binding domains, Mg2+ binding domains, among others), nucleotide-binding domains, regulatory domains, localization domains, kinase domains, phosphatase domains, protease domains, transferase domains, transporter domains, inhibitor domains, activator domains, extracellular domains, transmembrane domains, cytoplasmic domains, drug-binding domains, antibody fragment crystallizable domains, antibody variable domains, immunoglobulin domains, antibody-like domains, linker domains, catalytic domains, basic leucine zipper domains, cadherin repeat domains, NLRP3 domains (e.g., NACHT domains, LRR domains, and/or PYD domains), fibronectin domains, MHC class I protein domains, MHC class II protein domains, death effector domains, EF hand domains, zinc finger DNA binding domains, phosphotyrosine-binding domains, pleckstrin homology domains, Src homology 2 domains, and ADAR1 or ADAR2 Z-DNA binding domains or deaminase domains, among others. One of skill in the art would understand that other transgenes encoding protein domains may also be used in conjunction with the present invention, so long as the protein domains can function independently of the rest of their protein chain.

The transgenes suitable for expression using the vectors described herein may include polynucleotides encoding wild-type proteins and/or polypeptides. Alternatively, the transgenes may include polynucleotides encoding proteins and/or polypeptides that include one or more amino acid substitutions, such as one or more conservative amino acid substitutions (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions) relative to the wild-type polypeptide.

The transgenes suitable for expression via the vectors described herein may also encode a synthetic polypeptide including amino acid sequences of interest.

The transgenes suitable for expression via the vectors described herein may also encode proteins, protein domains, or polypeptides useful in a variety of applications including, but not limited to identification and development of new therapeutic agents, recombinant protein expression for cell-based functional assays, and protein production for crystallography applications, among others.

### Regulatory Elements

The regulatory elements are components of delivery vehicles used to facilitate nucleic acid molecule entry, replication, and/or expression in a host cell. The regulatory elements may be viral regulatory elements, which may optionally be baculoviral regulatory elements. For example, the viral regulatory elements may be the baculovirus homologous region (hr1) transcription enhancer. Other non-limiting examples of regulatory elements include the Tn7L promoter and terminator, Tn7R promoter and terminator, 39K promoter, IE1 terminator, T7 terminator, among others. The baculoviral regulatory elements may be from baculovirus or they may be heterologous sequences identified from other genomic regions. One skilled in the art would also appreciate that as other viral regulatory elements are identified, these may be used with the nucleic acid molecules described herein.

The vectors of the present invention may include an origin of replication (ori) sequence to enable replication of the vector in a host cell (e.g., a bacterial cell, an invertebrate cell, or a mammalian cell). Exemplary bacterial ori sequences include, but are not limited to ColE1, pMB1, pSC101, R6K, pUC, pBR322 and p15A ori sequences. The vectors of the instant invention may be replicated using techniques well known in the art.

The vectors of the present invention may further include 5′ and 3′ UTR sequences capable of directing and regulating transcription and/or translation. The 5′ UTR may include regulatory nucleic acid sequences important for the control of transcription and/or translation. Such sequences may modulate polyadenylation, translation efficiency, and mRNA localization and stability. Non-limiting examples of 3′ UTR regulatory sequences include enhancers, terminators (e.g. IE1 terminator, rrnB terminator), silencers, AU-rich elements, and microRNA recognition elements. Non-limiting examples of 3′ UTR enhancers include the Woodchuck Hepatitis Virus Posttranscriptional Regulatory Element (WPRE) enhancer. Non-limiting examples of 3′ UTR terminator sequences include the bovine growth hormone (bGH) and simian virus 40 (SV40) terminators. The vectors of the present invention may further include sequences encoding 2A self-cleaving peptides that facilitate the expression of multiple polypeptides from a single promoter.

### Selectable Markers

The vectors suitable for use with the present invention may also include nucleic acid sequences encoding one or more selectable markers, such as antibiotic resistance genes for selection of cells containing such a vector. Examples of suitable markers for use with the vectors described herein are genes that encode resistance to antibiotics, such as ampicillin, gentamycin, chloramphenicol, carbenicillin, kanamycin, nourseothricin, tetracycline, zeocin, streptomycin, and spectinomycin, among others. One of skill in the art would recognize that other selectable markers may also be used in conjunction with the present invention.

### Translocation Sequences

The recombinant vectors suitable for use with the compositions and methods described herein may also include translocation sequences (e.g., translocation sites) important for the insertion of transgenes and associated sequences into the vector. Non-limiting examples of translocation sites include the transposon 7 (Tn7) Tn7R and Tn7L sequences. One of skill the art would understand that other translocation sequences may be employed within the scope of the present invention.

### Baculovirus

The recombinant vectors of the invention may be used in such a way as to facilitate the production of viral particles capable of expressing recombinant proteins in both mammalian and insect cells or to allow for transient protein expression directly from the vector (e.g., plasmid) without the need for the production of virus.

The recombinant vectors for use in the present invention may be based on various viral genomes, including, but not limited to, Bombyx mori nuclear polyhedrosis virus, Orgyia pseudotsugata mononuclear polyhedrosis virus, Trichoplusia ni mononuclear polyhedrosis virus, Helioththis zea baculovirus, Lymantria dispar baculovirus, Cryptophlebia leucotreta granulosis virus, Penaeusmonodon-type baculovirus, Plodia interpunctella granulosis virus, Mamestra brassicae nuclear polyhedrosis virus, Autographa Californica nuclear polyhedrosis virus, or Buzura suppressaria nuclear polyhedrosis virus. Procedures for the production of baculovirus modified with heterologous genetic elements are well known in the art and can be found in, for example, Pfeifer et al., Gene 188:183-90 (1997), Clem et al., J Virol 68:6759-62, (1994), the disclosures of which are herein incorporated by reference.

### Host Cells

Cells that may be used in conjunction with the compositions and methods described herein include cells capable of expressing a transgene from the recombinant vector of the present invention. For example, one type of cell that can be used in conjunction with the compositions and methods described herein is a mammalian cell. Non-limiting examples of mammalian cells include primary cells (e.g., human, mouse, rat, or porcine primary cells, among others) or cell lines derived from human, mouse, rat, porcine, or other mammals. The mammalian cells for use with the present invention may be obtained or derived from any type of tissue including but not limited to liver, kidney, heart, skeletal muscle, smooth muscle, pancreatic, intestinal, bone, nervous system, blood, connective, adipose, skin, cervix, immune cells, tumor cells, and undifferentiated tissues, among others.

Another type of cell that can be used in conjunction with the compositions and methods described herein is an insect cell. Common and non-limiting examples of insect cell expression systems include Spodoptera frugiperda SF9 cells, mimic SF9 cells, SF21 cells, Trichoplusia ni BTI-TN-5B1-4 cells (also known as High Five cells), and Drosophila melanogasterS2 cells, among others. Insect cells may be wild-type insect cells or may be optimized through genetic engineering for recombinant protein expression. Such optimization strategies may be tailored to produce recombinant proteins having desirable properties for specific applications and may include engineering glycosylation profiles of insect cells, optimizing protein expression levels, transfection and/or transduction strategies, dosing, and protein purification and concentration, among others. Optimization strategies for insect host cells are described in detail in Gowder, S. J. T. (2017, New Insights into Cell Culture Technology, Chapter 2. IntechOpen, which is herein incorporated by reference. Recombinant protein expression using the vectors of the present invention may also be tailored for expression in insect larvae.

### Methods for Vector Delivery to Host Cells

Techniques that can be used to introduce a vector of the instant invention into a host cell are well known in the art. For example, electroporation can be used to permeabilize target cells by the application of an electrostatic potential to the cell of interest. Target cells, such as mammalian or insect cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA including the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

According to the methods and compositions of the present invention, recombinant viral particles can be introduced directly to the host cell by contacting the host cell in culture with a virus harboring the recombinant vector described herein. Upon contact with the host cell, the virus will attach to the host cell surface by specific interactions between viral capsid proteins and cell surface receptors on the host cell, resulting in endocytosis of the viral particles and cell entry. Within the cytoplasm, the viral particle will shed its capsid and release the viral genome into the host cell. Once the viral genome is exposed, its sequence may be transcribed into mRNA for protein expression or the viral genome may be replicated if the host cell is permissive to viral replication.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: Construction of Recombinant Vector for Protein Expression in Insect and Mammalian Cells

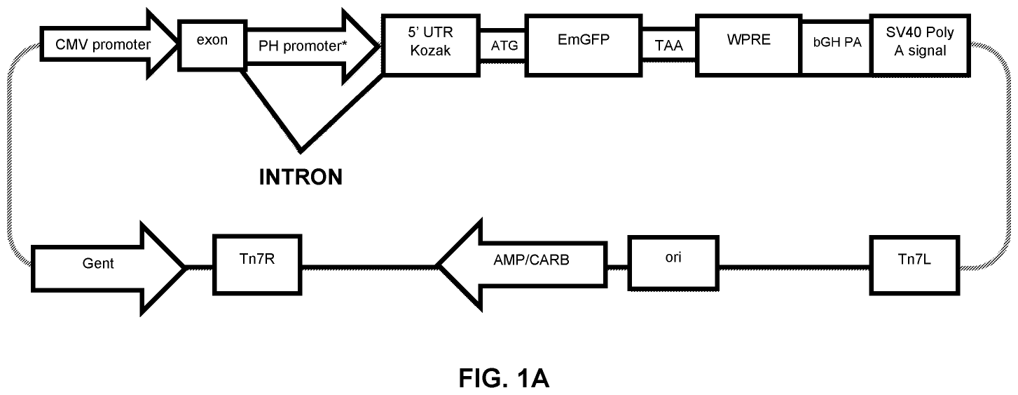

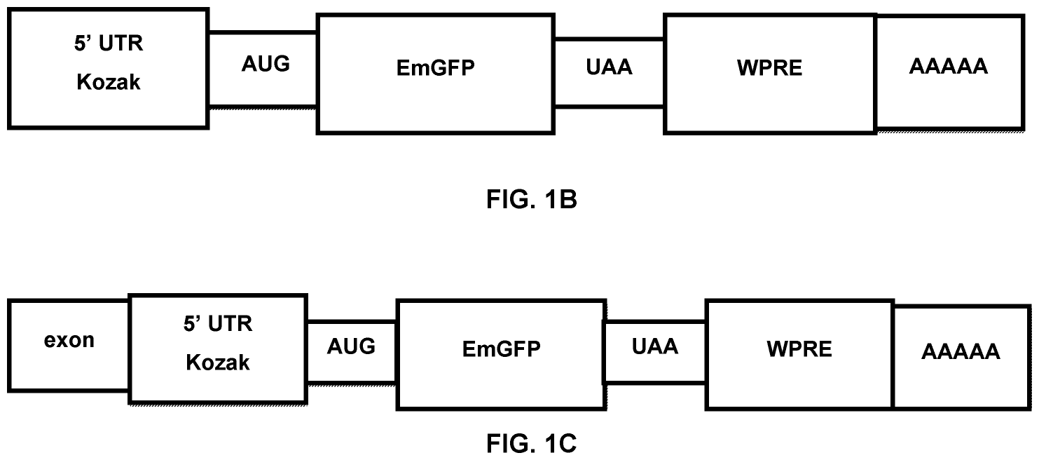

An expression vector was constructed to enable recombinant protein expression in insect and mammalian cells. A vector design was selected in which expression of a transgene was facilitated in both cell types by integrating mammalian cell-competent and insect cell-competent promoters in a unique design. As shown in FIG. 1A, one exemplary vector included, in the 5′ to 3′ direction, a cytomegalovirus (CMV) enhancer/promoter, a non-coding mini-exon, an artificial intron including a polyhedrin (PH) promoter flanked on the 5′ end with a splice donor sequence and on the 3′ end by a splice branch point, a polymyrimidine tract, and a 3′ splice acceptor sequence, followed by a 5′ untranslated region (5′ UTR) harboring a Kozak sequence, a start codon (AUG), a sequence encoding the transgene (e.g., emerald GFP (emGFP)), a stop codon (e.g., TAA), a 3′ untranslated region (3′ UTR) including a Woodchuck Hepatitis Virus Posttranscriptional Regulatory Element (WPRE), as well as bovine growth hormone (bGH) and simian virus 40 (SV40) terminator sequences. Additionally, the vector contained nucleic acid sequences encoding ampicillin (AMP/CARB) and gentamicin (Gent) antibiotic resistance genes, an E. coli origin of replication (ori), as well as two translocation sites Tn7L and Tn7R. A nucleic acid sequence contained within the exemplary vector above includes the CMV enhancer/promoter, non-coding mini-exon, artificial intron harboring the PH promoter, emGFP transgene, and the WPRE sequence and is provided below:

(SEQ ID NO: 1)
GCGTTACATAACTTACGGTAAATGGCCCGCCTGGCTGACCGCCCAACGAC
CCCCGCCCATTGACGTCAATAATGACGTATGTTCCCATAGTAACGCCAAT
AGGGACTTTCCATTGACGTCAATGGGTGGAGTATTTACGGTAAACTGCCC
ACTTGGCAGTACATCAAGTGTATCATATGCCAAGTACGCCCCCTATTGAC
GTCAATGACGGTAAATGGCCCGCCTGGCATTATGCCCAGTACATGACCTT
ATGGGACTTTCCTACTTGGCAGTACATCTACGTATTAGTCATCGCTATTA
CCATGGTGATGCGGTTTTGGCAGTACATCAATGGGCGTGGATAGCGGTTT
GACTCACGGGGATTTCCAAGTCTCCACCCCATTGACGTCAATGGGAGTTT
GTTTTGGCACCAAAATCAACGGGACTTTCCAAAATGTCGTAACAACTCCG
CCCCATTGACGCAAATGGGCGGTAGGCGTGTACGGTGGGAGGTCTATATA
AGCAGAGCTCGTTTAGTGAACCGTCAGATCACTAGAAGCTTTATTGCGGT
AGTTTATCACAGTTAAATTGCTAACGCAGTCAGTGCTTCTGACACAACAG
TCTCGAACTTAAGCTGCAGAAGTTGGTCGTGAGGCACTGGGCAGTAAGTA
TCATAGATCATGGAGATAATTAAAATGATAACCATCTCGCAAATAAATAA
GTATTTTACTGTTTTCGTAACAGTTTTGTAATAAAAAAACCTATAAATAT
TCCGGATTATTCATACCGTCCCACCATCGGGCGCCTTACTGAATCCACTT
TGCCTTTCTCTCCACAGGCTAGCATGGTGAGCAAGGGCGAGGAGCTGTTC
ACCGGGGTGGTGCCCATCCTGGTCGAGCTGGACGGCGACGTAAACGGCCA
CAAGTTCAGCGTGTCCGGCGAGGGCGAGGGCGATGCCACCTACGGCAAGC
TGACCCTGAAGTTCATCTGCACCACCGGCAAGCTGCCCGTGCCCTGGCCC
ACCCTCGTGACCACCTTGACCTACGGCGTGCAGTGCTTCGCCCGCTACCC
CGACCACATGAAGCAGCACGACTTCTTCAAGTCCGCCATGCCCGAAGGCT
ACGTCCAGGAGCGCACCATCTTCTTCAAGGACGACGGCAACTACAAGACC
CGCGCCGAGGTGAAGTTCGAGGGCGACACCCTGGTGAACCGCATCGAGCT
GAAGGGCATCGACTTCAAGGAGGACGGCAACATCCTGGGGCACAAGCTGG
AGTACAACTACAACAGCCACAAGGTCTATATCACCGCCGACAAGCAGAAG
AACGGCATCAAGGTGAACTTCAAGACCCGCCACAACATCGAGGACGGCAG
CGTGCAGCTCGCCGACCACTACCAGCAGAACACCCCCATCGGCGACGGCC
CCGTGCTGCTGCCCGACAACCACTACCTGAGCACCCAGTCCGCCCTGAGC
AAAGACCCCAACGAGAAGCGCGATCACATGGTCCTGCTGGAGTTCGTGAC
CGCCGCCGGGATCACTCTCGGCATGGACGAGCTGTACAAGTAAGCGGCCG
CAATCAACCTCTGGATTACAAAATTTGTGAAAGATTGACTGGTATTCTTA
ACTATGTTGCTCCTTTTACGCTATGTGGATACGCTGCTTTAATGCCTTTG
TATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCC
TATTGCCACGGCGGAACTCATCGCCGCCTGCCTTGCCCGCTGCTGGACAG
GGGCTCGGCTGTTGGGCACTGACAATTCCGTGGTGTTGTCGGGGAAGCTG
ACGTCCTTTCCATGGCTGCTCGCCTGTGTTGCCACCTGGATTCTGCGCGG
GACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGC
CCTCAGACGAGTCGGATCTCCCTTTGGGCCGCCTCCCCGCCTGGGCCCGT
TTAAACCCGCTGATCA

In insect cells, the mRNA of the transgene (e.g., emGFP) is transcribed from the strong baculoviral PH promotor, and is accomplished by the baculoviral RNA polymerase. There is no transcription from the CMV promoter in insect cells; all transcription originates from the PH promoter, which produces an mRNA transcript as shown in FIG. 1B. In mammalian cells, mRNA is transcribed from the CMV promoter by mammalian RNA polymerase II. Reciprocally, the CMV promoter is inactive in insect cells, while the PH promoter is inactive in mammalian cells. Thus, the transcript produced includes the elements shown in FIG. 1C. The PH promoter and the intron include one or more of start and stop codons which inhibit the translation of a continuous reading frame in mammalian cells. However, the intron is subjected to splicing during mRNA maturation in the mammalian cell which removes the PH promoter-containing intron together with all associated open reading frames and stop codons. Thus the 5′ UTR of all mRNAs transcribed from the CMV promoter includes a short 5′ noncoding mini-exon which has little or no effect on protein translation, in addition to all the same elements of the mature mRNA

### Example 2: Expression of Recombinant Proteins in Insect and Mammalian Cells

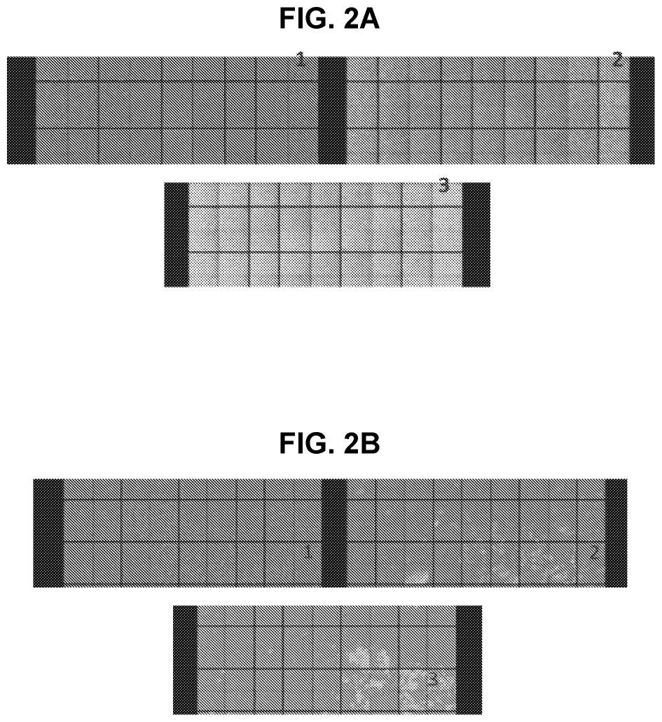

In order to demonstrate the efficacy of the recombinant vector to drive transgene expression in both insect and mammalian cells, separate cell culture assays were performed on insect SF9 cells and mammalian HEK293F cells in the presence of varying doses of viral particles harboring a recombinant vector encoding the emGFP gene. First, a recombinant plasmid was produced by preparing a donor plasmid containing an expression cassette harboring a GFP transgene as described in Example 1. The donor plasmid was subsequently transformed into the DH10Bac E. coli cell line containing a helper plasmid that produces a Tn7 transposase enzyme, and a plasmid containing baculoviral DNA (e.g., a bacmid) having a mini-attTn7 site within the open reading frame of the β-galactosidase gene. Following transposition-mediated incorporation of the expression cassette from the donor plasmid into the bacmid, the newly formed recombinant plasmid was artificially selected, amplified, and purified from LacZ-negative E. coli cells on the basis of its large molecular size (around 130 kb). SF9 cell cultures were subsequently transfected with the isolated recombinant plasmids for viral amplification. Following 2-3 generations of virus production, cultured SF9 cells (FIG. 2A) and HEK293F cells (FIG. 2B) were infected with 200 μL or 400 μL of recombinant viral particles and allowed to incubate for 16 hours. A separate control group did not receive a viral dose. As seen in FIGS. 2A-2B, robust GFP expression was observed in both insect and mammalian cell cultures. These results indicate that virus produced from the recombinant vector described herein is capable of driving protein expression in insect and mammalian cells.

### Example 3: Splicing of the Artificial Intron Encoded by a Recombinant Vector in Mammalian Cells

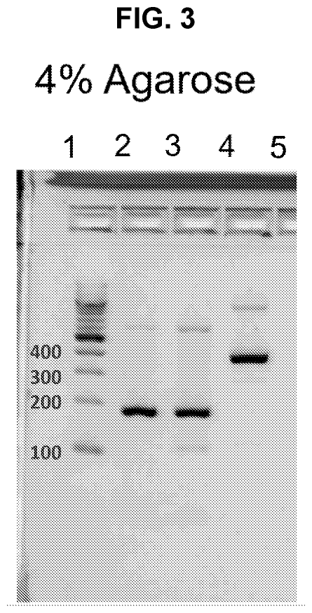

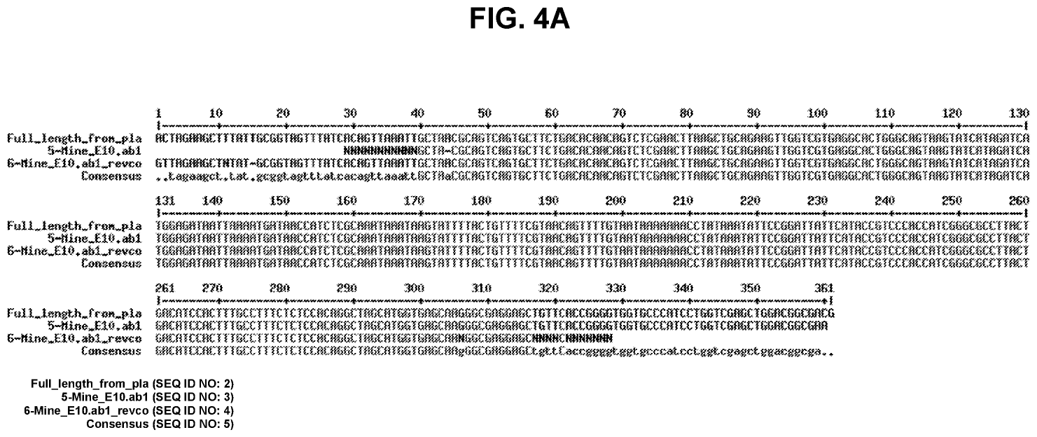

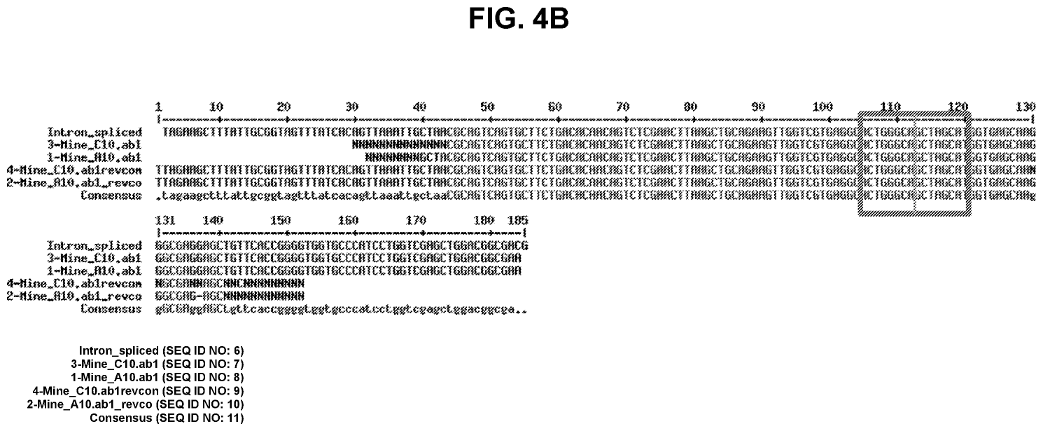

To confirm the removal of the artificial intron containing a PH promoter from mRNA transcripts in mammalian cells via a splicing event, RT-PCR experiments were performed. HEK293 cells were subsequently infected with the recombinant vector harboring a GFP transgene as described in Example 2, and incubated for 16 hours. Total RNA was extracted from about 2 million cells using Qiagen RNeasy kit. Reverse transcription was performed using Superscript IV (Invitrogen) and gene-specific primers or an oligo-dT/random hexamer mix followed by 30 cycles of PCR amplification using nested primers. As a control, PCR amplification was performed from the vector. Expected length of the spliced product was 186 bp, whereas unspliced precursor (as in the plasmid) was 357 bp long. As shown in FIG. 3, both RT-PCR reactions with gene-specific (lane 2) and oligo-dT/N6 (lane 3) were spliced, and their lengths were about 180-190 bp on the gel, as expected if the intron was removed. Amplification of the plasmid produced a 350 bp product, as expected if the intron was present (lane 4). PCR products were Sanger sequenced (Genewiz) which confirmed the accuracy of the splicing (FIGS. 4A-4B). Thus, these findings indicated that a vector design strategy incorporating a PH promoter into an artificial intron downstream of a CMV promoter and a non-coding mini-exon allowed for successful removal of the PH promoter in mammalian cells through a splicing event.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

What is claimed is:

1. A recombinant DNA vector comprising in a 5′ to 3′ direction:
(a) a mammalian cell-competent promoter;
(b) a non-coding exon operably linked to an artificial intron, the artificial intron comprising a splice donor sequence, an insect cell-competent promoter, a splice branch point, a polypyrimidine tract, and a splice acceptor sequence; and
(c) one or more transgenes operably linked to the mammalian cell-competent promoter and to the insect cell-competent promoter.

2. The vector of claim 1, wherein the mammalian cell-competent promoter is selected from the group consisting of a cytomegalovirus (CMV) enhancer/promoter, simian virus 40 (SV40) promoter, CAG promoter, elongation factor 1 (EF1-α) promoter, phosphoglycerate kinase 1 (PGK1) promoter, β-actin promoter, early growth response 1 (EGR1) promoter, eukaryotic translation initiation factor 4A1 (eIF4A1) promoter, glyceraldehyde 3-phosphate dehydrogenase (GAPDH) promoter, human immunodeficiency virus long terminal repeat (HIV LTR) promoter, Adenoviral promoter, and Rous Sarcoma Virus (RSV) promoter.

3. The vector of claim 2, wherein the mammalian cell-competent promoter is a CMV enhancer/promoter.

4. The vector of claim 1, wherein the insect cell-competent promoter is selected from a group consisting of a polyhedrin (PH) promoter, heat shock protein (HSP) promoter, p6.9 promoter, p9 promoter, p10 promoter, actin 5c (Ac5) promoter, Orgyia pseudotsugata multicapsid nuclear polyhedrosis virus immediate early-1 (OpIE1) promoter, Orgyia pseudotsugata multicapsid nuclear polyhedrosis virus immediate early-2 (OpIE2) promoter, and an immediate early-0 (IE0) promoter.

5. The vector of claim 4, wherein the insect cell-competent promoter is a PH promoter.

6. The vector of any one of claims 1-5, wherein the vector further comprises a 5′ untranslated region (5′ UTR) with a Kozak sequence.

7. The vector of any one of claims 1-6, wherein the vector further comprises a 3′ untranslated region (3′ UTR).

8. The vector of claim 7, wherein the 3′ UTR comprises an enhancer sequence.

9. The vector of claim 8, wherein the enhancer sequence is a Woodchuck Hepatitis Virus Posttranscriptional Regulatory Element (WPRE).

10. The vector of any one of claims 7-9, wherein the 3′ UTR further comprises one or more terminator sequences.

11. The vector of claim 10, wherein the one or more terminator sequences is selected from a group consisting of a bovine growth hormone (bGH) terminator sequence and a simian virus 40 (SV40) terminator sequence.

12. The vector of any one of claims 1-11, wherein the vector further comprises one or more nucleic acid sequences encoding one or more selectable marker genes.

13. The vector of claim 12, wherein the one or more selectable marker genes are selected from the group consisting of an ampicillin resistance gene, gentamycin resistance gene, carbenicillin resistance gene, chloramphenicol resistance gene, kanamycin resistance gene, nourseothricin resistance gene, tetracycline resistance gene, zeocin resistance gene, streptomycin resistance gene, and spectinomycin resistance gene.

14. The vector of any one of claims 1-13, wherein the vector further comprises two translocation elements.

15. The vector of claim 14, wherein the two translocation elements are bacterial transposon Tn7R and Tn7L translocation elements.

16. The vector of any one of claims 1-15, wherein the one or more transgenes are mammalian genes.

17. The vector of any one of claims 1-15, wherein the one or more transgenes are insect genes.

18. A method of expressing a recombinant protein in a host cell, the method comprising contacting the host cell with the vector of any one of claims 1-17; and expressing the recombinant protein in the host cell.

19. The method of claim 18, wherein the host cell is a mammalian cell.

20. A method of expressing a recombinant protein in a host cell, the method comprising contacting the host cell with a recombinant virus produced using the vector of any one of claims 1-17; and expressing the recombinant protein in the host cell.

21. The method of claim 20, wherein the host cell is an insect cell or a mammalian cell.
